(19) United States
(12) Patent Application Publication
(10) Pub. No.: US 2022/0218415 A1
(43) Pub. Date: Jul. 14, 2022

(54) SYSTEM AND METHOD FOR TREATMENT OF HUMAN STONES

(71) Applicant: LISA Laser Products GmbH, Katlenburg-Lindau (DE)

(72) Inventors: Samir Lamrini, Goettingen (DE); Thorsten Muehlke, Goettingen (DE); Bjoern Christoph, Bodensee (DE)

(21) Appl. No.: 17/576,125

(22) Filed: Jan. 14, 2022

Related U.S. Application Data
(60) Provisional application No. 63/137,557, filed on Jan. 14, 2021.

(51) Int. Cl. A61B 18/26

(52) U.S. Cl. CPC A61B 18/26; A61B 2018/2065

(57) ABSTRACT

A laser lithotripsy system includes a thulium-based laser that, upon activation, selectively produces a continuous wave of laser light with a first wavelength or uniformly spaced, intermittent pulses of laser light with the first wavelength. The system further includes a second laser that, upon activation, produces laser light with a second wavelength, which is shorter than the first wavelength. The system includes an optical detector positioned to receive light emitted by a target in response to the target being impacted by the light produced by the second laser, and a controller communicatively coupled to both the optical detector and the first laser such that the controller selectively activates and deactivates the first laser based on one or more measured characteristics of the light emitted by the target and received by the optical detector.

### BACKGROUND

### Technical Field

The present disclosure relates to medical devices and methods of treatment. More specifically, the disclosure relates to systems and methods for treating human stones.

### Description of the Related Art

Human stones may develop within a human body and cause symptoms, such as pain. One type of human stone is a kidney stone. Kidney stone disease, also known as urolithiasis, is when a solid piece of material (kidney stone) develops in the urinary tract. Kidney stones typically form in the kidney and leave the body in the urine stream. A small kidney stone may pass without causing symptoms, however if a kidney stone grows to more than 5 millimeters, it can cause blockage of the ureter, resulting in severe pain. When a human stone causes no symptoms, no treatment is needed. However, larger human stones may require procedures such as ureteroscopy for removal.

Ureteroscopy is a procedure in which a urologist positions an endoscope proximate a target area for treatment within a patient's body. Using a laser, the urologist fragments the kidney stone into smaller pieces and retracts the fragments with a basket. Known ureteroscopy treatment utilizes a holmium, e.g., a Holmium:yttrium-aluminium-garnet (Ho:YAG), laser to break up kidney stone fragments in a procedure known as lithotripsy.

### BRIEF SUMMARY

The use of a high energy source, such as a laser, within a human body, e.g., in the upper urinary tract, may cause severe damage. Improper positioning of the tip of the laser delivery system, e.g., the laser fiber, may result in tissue burn, urinary tract perforation, or kidney/bladder tissue damage. Known laser lithotripsy systems are visually controlled by the surgeon performing the procedure. The surgeon's view of the target area for the laser may be obscured or affected, e.g., by stone dust or bleeding. Activation of the laser while the target area is obscured may result in activation of the laser when tissue, rather than a human stone, is in the target area.

Accordingly, systems and methods of identifying and distinguishing between tissue and human stones during laser lithotripsy may result in improved outcomes for treatment of patients suffering from urolithiasis.

According to one aspect of the disclosure, a laser lithotripsy system includes a first laser that, upon activation, produces laser light with a first wavelength. The first laser includes a first activation mode and a second activation mode. When the first laser is in the first activation mode the first laser produces a continuous wave of laser light with the first wavelength, and when the first laser is in the second activation mode the first laser produces uniformly spaced, intermittent pulses of laser light with the first wavelength.

The system further includes a second laser that, upon activation, produces light with a second wavelength, which is shorter than the first wavelength. The system includes a first optically powered surface positioned to receive both the light from the first laser and the light from the second laser, and the optically powered surface transmits at least 90% of the light received from the first laser and reflects at least 90% of the light received from the second laser such that the transmitted light from the first laser is superimposed with the reflected light from the second laser.

The system includes a waveguide, e.g., a glass fiber, positioned to receive the superimposed light from the first and second lasers and guide the superimposed light to a target, an optical detector positioned to receive light emitted by the target and measure one or more characteristics of the received light emitted by the target, and a controller. The controller is communicatively coupled to both the optical detector and the first laser such that the controller activates the first laser when the one or more measured characteristics are within a predetermined range of values and prevents activation of the first laser when the one or more measured characteristics are outside of the predetermined range of values.

According to another aspect of the disclosure, a method of treatment includes activating an excitation laser to produce laser light and guiding the produced laser light to a target via a waveguide. The method includes capturing light emitted from the target as a result of the laser light produced by the excitation laser impacting the target, guiding the captured light emitted from the target to an optical detector via the waveguide, and measuring one or more characteristics of the captured light emitted by the target and guided to the optical detector. The method further includes comparing the one or more measured characteristics to a predetermined set of values for each of the one or more measured characteristics, and activating a therapeutic laser when the one or more measured characteristics are within the respective predetermined set of values for each of the one or more measured characteristics, wherein activating the therapeutic laser produces a continuous wave of laser light when the therapeutic laser is in a first activation mode, and activating the therapeutic laser produces uniformly spaced, intermittent pulses of laser light when the therapeutic laser is in a second activation mode

According to another aspect of the disclosure, a method of treating human stones includes activating an excitation laser to produce laser light and guiding the produced laser light to a distal end of a waveguide where the produced laser light exits the waveguide. The method includes moving the distal end of the waveguide such that the produced laser light exits the waveguide and impacts a human stone, capturing light emitted from the human stone as a result of the laser light produced by the excitation laser impacting the human stone, and guiding the captured light emitted from the human stone to an optical detector via the waveguide.

The method further includes measuring one or more characteristics of the captured light emitted by the human stone and guided to the optical detector, determining whether the captured light was emitted by a human stone based on the measured one or more characteristics of the captured light, after determining the captured light was emitted by a human stone, activating a therapeutic laser thereby producing either a continuous wave of laser light with a first wavelength or uniformly spaced, intermittent pulses of laser light with the first wavelength, and guiding the continuous wave of laser light to the distal end of the waveguide where the continuous wave of laser light exits the waveguide and impacts the human stone with the continuous wave of light.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not necessarily drawn to scale, and some of these elements may be arbitrarily enlarged and positioned to improve drawing legibility. Further, the particular shapes of the elements as drawn, are not necessarily intended to convey any information regarding the actual shape of the particular elements, and may have been solely selected for ease of recognition in the drawings.

FIG. 1 is a side, schematic view of a therapeutic laser system, according to an embodiment.

FIG. 2 is a side, schematic view of a portion of the therapeutic laser system illustrated in FIG. 1 in use, and the system is targeting a first human stone.

FIG. 3 is a side, schematic view of the portion of the therapeutic laser system illustrated in FIG. 2 in use, and the system is targeting soft tissue.

FIG. 4 is a side, schematic view of the portion of the therapeutic laser system illustrated in FIG. 2 in use, and the system is targeting a second human stone.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with therapeutic laser systems have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment,” “an embodiment,” or “an aspect of the disclosure” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its broadest sense, that is as meaning “and/or” unless the content clearly dictates otherwise.

Recitation of ranges of values herein are merely intended to serve as a shorthand method of referring individually to each separate value falling within the range including the stated ends of the range, unless otherwise indicated herein, and each separate value is incorporated into the specification as if it were individually recited herein.

Aspects of the disclosure will now be described in detail with reference to the drawings, wherein like reference numbers refer to like elements throughout, unless specified otherwise. Certain terminology is used in the following description for convenience only and is not limiting. The term “plurality”, as used herein, means more than one. The terms “a portion” and “at least a portion” of a structure include the entirety of the structure.

The headings and Abstract of the Disclosure provided herein are for convenience only and do not interpret the scope or meaning of the embodiments.

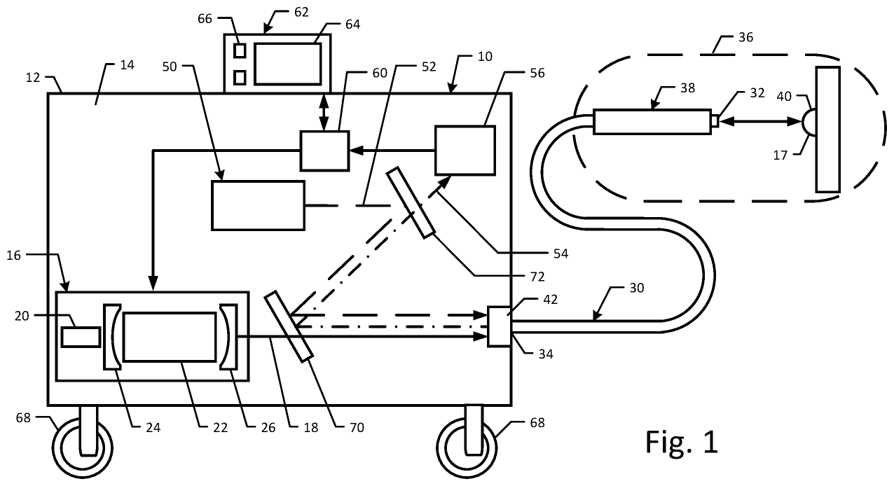

Referring to FIG. 1, a therapeutic laser system 10 includes a housing 12 that selectively encloses an internal cavity 14 formed by the housing 12. The system 10 includes a first laser 16 (also referred to herein as a therapeutic laser). The first laser 16, upon activation, produces a focused beam of light 18 (i.e., laser light) with a first wavelength. Energy, typically via a pump 20, is supplied to a lasing medium 22. The supplied energy causes electrons within atoms of the lasing medium 22 to become “excited” and increase their energy level. Once these “excited” electrons return to their “non-excited” ground state (or energy level), energy is released in the form of photons. These photons are reflected back and forth through the lasing medium 22 by a pair of mirrors. A first mirror 24 of the pair of mirrors is a total reflector, which reflects all of the photons that impact the first mirror 24 back toward the lasing medium 22. A second mirror 26 of the pair of mirrors is a partial reflector, which reflects a portion of the photons that impact the second mirror 26 back toward the lasing medium 22, while allowing a focused beam of the photons to pass through the second mirror 26 and exit the laser 16, thereby forming the focused beam of light 18.

Holmium-based lasers are known for their use in a number of therapeutic applications. A holmium-based laser includes holmium as the lasing medium. However, there are several drawbacks to holmium-based lasers. Medical holmium-based lasers are pumped by flashlamps, which operate in a pulsed mode, thus holmium-based lasers produce a pulsed beam of laser light that includes intervals of high peaks of power separated by intervals of relatively low (or no) power. For example, a holmium-based laser may operate at 30 Hertz (Hz), such that the beam of light produced over one second includes 30 peaks of power separated by 30 intervals of low (or no) power of equal length. Additionally, the frequency of holmium-based lasers is high enough to cause retropulsion (i.e., movement) of human stones that are impacted by beam of light created by a holmium-based laser. For example, a Holmium:yttrium-aluminium-garnet (Ho:YAG) laser produces a beam of light with a wavelength of 2,100 nanometers (nm).

According to one embodiment, the focused beam of light 18 produced by the first laser 16 of the therapeutic laser system 10 may be in the form of a continuous wave. As used herein a continuous wave is an alternative form to a pulsed laser as described above. Rather than regularly spaced intervals of peaks of high power spaced by intervals of low (or no) power, a continuous wave laser maintains a steady output of power over an amount of time (e.g., a second or greater) until the laser producing the continuous wave is affirmatively deactivated (e.g., by a user or a controller). A continuous wave laser may be activated and deactivated, but the intervals between such activations are not of equal length.

The use of a continuous wave of the focused beam of light 18 results in less energy being needed to achieve the same therapeutic result as would be required to operate a pulsed beam. Additionally, the continuous wave of the focused beam of light 18 transfers less heat to a target impacted by the focused beam of light 18.

According to one embodiment, the focused beam of light 18 produced by the first laser 16 of the therapeutic laser system 10 may have a wavelength that is shorter than 2,100 nm. For example, a thulium:yttrium-aluminium-garnet (Tm:YAG) laser produces a focused beam of light with a wavelength of 2,010 nm.

A thulium laser may be pumped by laser diodes, which may operate at a higher wall-plug efficiency compared to the flashlamps of a holmium laser, thus result in a higher efficiency for a thulium laser. Thulium lasers, however, present challenges related to their engineering and construction. Specifically, the optical focusing design related to a thulium laser used in the therapeutic laser system 10 may be more complex and/or expensive than a holmium laser, as the thulium laser may be operable in both a continuous wave mode and a pulsed mode. Thus, a thulium laser may require the development of a complex focusing lens system as well as a resonator/cavity design that each meet the operating criteria for multiple modes of laser operation. Thulium lasers may produce laser light with a wavelength between 1800 nm and 2200 nm.

The focused beam of light 18 produced by the first laser 16 is guided to a target 17. As shown in the illustrated embodiment, the system 10 may include a waveguide 30 (e.g., a laser fiber), with an internal cavity which guides the focused beam of light 18 along a length of the waveguide 30. The waveguide 30 may include a distal end 32 at which the focused beam of light 18 exits the internal cavity of the waveguide 30. The waveguide 30 may be flexible so that the distal end 32 is moveable (e.g., relative to a proximal end 34 of the waveguide 30 that is attached to the housing 14) to be positioned adjacent the target 17.

According to one embodiment, the target 17 may be located within a human body 36. For example, the target 17 may include one or more urinary stones within a patient's urinary tract. Thus, the system 10 may include an endoscope 38 (e.g., a cystoscope, a ureteroscope, a renoscope, a nephroscope, etc.) and the waveguide 30 may be sized to fit within the endoscope 38 during insertion of the endoscope 38 into the patient's body and advancement to the target 17.

During advancement of the endoscope 38 and the enclosed waveguide 30, the distal end 32 may be enclosed within an internal cavity of the endoscope 38, thus protecting the distal end 32 from damage (e.g., due to contact with body tissue). Upon arrival at the target 17, the waveguide 30 may be advanced within the endoscope 38 such that the distal end 32 is exposed as shown in the illustrated embodiment. The advancement of the waveguide 30 may help prevent the focused beam of light 18 from impacting and potentially damaging the endoscope 38.

With the distal end 32 of the waveguide 30 pointed at the target 17, activation of the first laser 16 results in the focused beam of light 18 impacting the target 17. According to one embodiment, the target 17 includes a human stone 40 (e.g., a urinary stone), and sustained impact of the focused beam of light 18 with the human stone 40 results in the human stone 40 breaking into multiple fragments, which due to their smaller size are easier to remove from the patient's body 36.

The system 10 may include a waveguide coupler 42, which couples the proximal end 34 of the waveguide 30 to the housing 12.

The system 10 may further include a second laser 50. As shown both the first laser 16 and the second laser 50 may be enclosed within the internal cavity 14 of the housing 12. The close proximity of the first laser 16 and the second laser 50 may result in smaller losses and thus better efficiency of the system 10. The second laser 50, upon activation, produces a focused beam of light 52 with a second wavelength.

According to one embodiment, the second laser 50 is an excitation laser (e.g., a green excitation laser that produces a focused beam of light 52 with a wavelength of 532 nm). An excitation laser, as used herein, refers to a laser suitable for use in a laser-induced fluorescence (LIF) application. LIF involves the excitation of an atom or molecule to a higher energy level upon the absorption of laser light, such as the focused beam of light 52 of the second laser 50. Some time after the absorption of the laser light the energy is released in the form of emission of light from the atom or molecule.

The system 10, according to one embodiment, directs the focused beam of light 52 of the second laser 50 to enter the waveguide 30, which then guides the focused beam of light 52 to the distal end 32 where the focused beam of light 52 exits the waveguide 30 and impacts the target 17. If both the first laser 16 and the second laser 50 are activated at the same time, the focused beam of light 18 and the focused beam of light 52 may coincide. It will be appreciated that the focused beams of light are shown as separate elements within the drawings for clarity purposes.

The second laser 50 may include a single mode (e.g., pulsed or continuous wave). According to one embodiment, the second laser 50 may include a plurality of modes (e.g., pulsed and continuous wave). The pulsed mode of the second laser 50 may include multiple settings with varied pulsed durations. According to one embodiment, the focused beam of light 52 of the second laser 50 has a second wavelength of between 500 nm and 600 nm, an output power of between 40-80 millijoules (mJ), a pulse duration of between 1 and 2 microseconds (μs), or any combination thereof.

Analyzing the reaction of the target 17 to the impact of the focused beam of light 52 of the second laser 50 may allow the target 17 to be identified or at least classified without direct, visual observation. For example, a human stone may emit a fluorescence signal with an amplitude that is higher (e.g., at least three times higher than an amplitude of a fluorescence signal of either urinary tract tissue or components of an endoscope.

A fluorescence signal 54 emitted by the target 17 in response to impact of the focused beam of light 52 of the second laser 50 may travel in the opposite direction of the focused beam of light 52 of the second laser 50, such that the fluorescence signal 54 enters the distal end 32 of the waveguide 30 and exits the proximal end 34 into the internal cavity 14 of the housing 12. Upon entry to the housing 12 the fluorescence signal 54 may be directed to an optical detector 56 of the system 10. The fluorescence signal 54 may have a third wavelength (or range of wavelengths) that is shorter than the first wavelength of the focused beam of light 18 and longer than the focused beam of light 52. According to one embodiment, the third wavelength is between 550 nm and 900 nm.

The optical detector 56 measures one or more characteristics of the fluorescence signal 54 emitted by the target 17. According to one embodiment, the one or more characteristics include an intensity of the fluorescence signal 54, a spectrum of the fluorescence signal 54, or both the intensity and the spectrum of the fluorescence signal 54. For example, the optical detector 56 may measure the amplitude, the wavelength, or both of the fluorescence signal 54.

A first, relatively low, amplitude may indicate that the fluorescence signal 54 is being emitted by human tissue (e.g., urinary tract tissue), thus indicating that the target 17 is human tissue. A second, relatively high, amplitude (for example at least twice the amplitude of the first amplitude) may indicate that the fluorescence signal 54 is being emitted by a human stone, thus indicating that the target 17 is a human stone. The measured one or more characteristics of the fluorescence signal 54 emitted by the target 17 may provide additional information, such as the main component of the target 17 (i.e., the specific type of human stone).

The system 10 may further include a controller 60 communicatively coupled to both the optical detector 56 and the first laser 16. The controller 60 receives data from the optical detector 56 identifying whether the target 17 is one that is meant to be impacted by the focused beam of light 18 (e.g., a human stone) or one that is not meant to be impacted by the focused beam of light 18 (e.g., tissue of the patient or a component of the endoscope 38). Upon receipt of data that identifies the target 17 as an object not meant to be impacted by the focused beam of light 18, the controller 60 prevents activation of the first laser 16 until data is received by the controller 60 that the target 17 is one that is meant to be impacted by the focused beam of light 18. Thus, according to one embodiment, the controller 60 is able to both activate the first laser 16 when the one or more measured characteristics of the fluorescence signal 54 are within a predetermined range of values (e.g., that identify the target 17 as a human stone) and prevent activation of the first laser 16 when the one or more measured characteristics are outside of the predetermined range of values (e.g., thus identifying the target 17 as human tissue, an endoscope, or an object other than a human stone).

The system 10 may further include a user interface 62 that includes a display 64, input controls 66, or both. The display 64 may show operational parameters of the system 10 including, but not limited to, the status (e.g., activated/not activated, continuous wave mode/pulse mode, etc.) of the first laser 16, the status of the second laser 50, the identification/classification of the target 17, etc. The input controls 66 may allow a user of the system 10 to change one or more of the operational parameters of the system 10 including, but not limited to, the status (e.g., activated/not activated, continuous wave mode/pulse mode, etc.) of the first laser 16, the status of the second laser 50, etc.

The system 10 may be mobile. As shown, the housing 12 may be mounted on wheels 68 so as to allow a user of the system 10 to change the location of the system 10.

The system 10 may include one or more optically powered elements (e.g., a lens, a mirror, etc.) that facilitate guiding the focused beam of light 18 and the focused beam of light 52 into the waveguide, and guiding the fluorescence signal 54 to the optical detector 56. As shown in the illustrated embodiment, the system 10 may include a first optically powered element 70 positioned within the internal cavity 14 of the housing 12 such that both the focused beam of light 18 and the focused beam of light 52 impact the first optically powered element 70.

According to one embodiment, the first optically powered element 70 is structured so as to be highly transmissive for light of the first wavelength and highly reflective for light of the second wavelength. As shown, the focused beam of light 18 from the first laser 16 passes through the first optically powered element 70 without significant alteration or losses. For example, according to one embodiment at least 90% of the focused beam of light 18 that impacts the first optically powered element 70 exits the first optically powered element 70 along the same direction with which it entered.

As shown, the focused beam of light 52 from the second laser 50 reflects off of the first optically powered element 70 without significant losses. For example, according to one embodiment at least 90% of the focused beam of light 52 that impacts the first optically powered element 70 reflects off of the first optically powered element 70 and is superimposed (or coincident with) the focused beam of light 52 as it exits the first optically powered element 70. According to one embodiment, the first optically powered element 70 is highly transmissive of light with a wavelength of between 2000 nm and 2200 nm, and the first optically powered element 70 is highly reflective of light with a wavelength between 500 nm and 900 nm.

The system 10 may include a second optically powered element 72 positioned within the internal cavity 14 of the housing 12 such that both the focused beam of light 52 and the fluorescence signal 54 impact the second optically powered element 72.

According to one embodiment, the second optically powered element 72 is structured so as to be highly transmissive for light of the third wavelength (i.e., the fluorescence signal 54) and highly reflective for light of the second wavelength (i.e., the focused beam of light 52 of the second laser 50). As shown, the fluorescence signal 54 passes through the second optically powered element 72 without significant alteration or losses. For example, according to one embodiment at least 90% of the fluorescence signal 54 that impacts the second optically powered element 72 exits the second optically powered element 72 and enters the optical detector 56.

As shown, the focused beam of light 52 from the second laser 50 reflects off of the second optically powered element 72 without significant losses. For example, according to one embodiment at least 90% of the focused beam of light 52 that impacts the second optically powered element 72 reflects off of the second optically powered element 72 and is guided toward the waveguide 30, for example via the first optically powered element 70. According to one embodiment, the second optically powered element 72 is highly transmissive of light with a wavelength of between 550 nm and 900 nm, and the second optically powered element 72 is highly reflective of light with a wavelength between 500 nm and 540 nm.

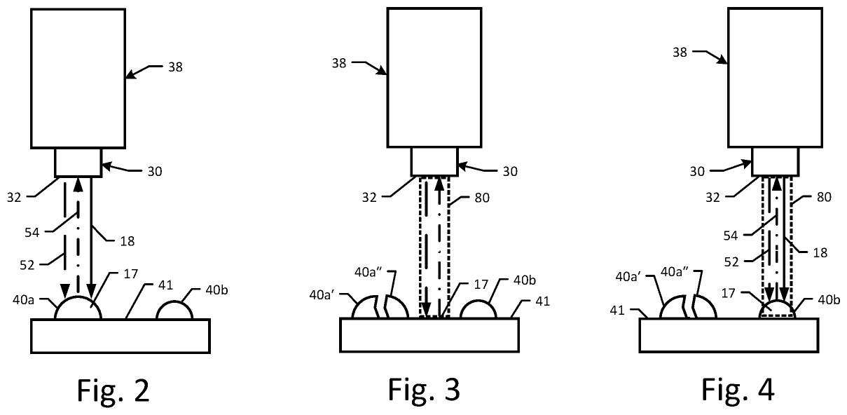

Referring to FIGS. 1 to 4 a method of treating human stones (e.g., urinary stones) includes activating the second laser 50 to produce the focused beam of light 52 and guiding the focused beam of light 18 to the distal end 32 of the waveguide 30 where the focused beam of light 18 exits the waveguide 30. As shown in FIG. 2, the method includes moving the distal end 32 of the waveguide 30 such that the focused beam of light 52 exits the waveguide 30 and impacts a first human stone 40a.

The method may further include capturing light emitted (e.g., the fluorescence signal 54) from the first human stone 40a as a result of the focused beam of light 52 impacting the first human stone 40a, and then guiding the captured light emitted from the first human stone 40a to the optical detector 56 via the waveguide 30.

The method may include measuring one or more characteristics of the fluorescence signal 54 and determining whether the fluorescence signal 54 was emitted by a human stone based on the measured one or more characteristics of the fluorescence signal 54. After determining the fluorescence signal 54 was emitted by a human stone, the method may include activating (e.g., via the controller 60) the first laser 16 thereby producing the focused beam of light 18 in the form of a continuous wave. The method further includes guiding the focused beam of light 18 to the distal end 32 of the waveguide 30 where the focused beam of light 18 exits the waveguide 30 and impacts the first human stone 40a.

The method may include impacting the first human stone 40a with the continuous wave of the focused beam of light 18 until the first human stone 40a breaks into multiple fragments (e.g., a first fragment 40a′ and a second fragment 40a″). The fragments may be discrete elements of a smaller size than the whole first human stone 40a. Breaking the first human stone 40a may include pulverizing the first human stone 40a such that at least portions of the first human stone 40a are reduced to dust.

After breaking the first human stone 40a into multiple fragments, moving one or more of the fragments, the distal end 32 of the waveguide 30, or both one or more of the fragments and the distal end 32 of the waveguide 30 such that the focused beam of light 52 of the second laser 50 exits the waveguide 30 and impacts a target other than one of the multiple fragments of the first human stone 40a.

As shown in FIG. 3, the system 10 may define a treatment space 80. The treatment space 80 may be the volume within which the focused beam of light 18 of the first laser 16 is effective at delivering the intended therapeutic treatment (e.g., fragmenting a human stone). According to one embodiment, the treatment space 80 extends out from the distal end 32 of the waveguide 30 (e.g., between 30 to 300 microns (μm)) and is bounded by perimeter that corresponds to a core diameter of the waveguide 30 (e.g., between 10-20 μm).

When the treatment space 80 is devoid of a human stone, the target 17 may be human tissue 41, a portion of the endoscope 38, or nothing at all (i.e., air) such that the fluorescence signal 54 may be minimal or absent entirely. Thus, the method may include attempting to capture the fluorescence signal 54 when the treatment space 80 is devoid of a human stone. If the fluorescence signal 54 is absent or of a relatively low amplitude so as to indicate that the treatment space 80 is devoid of a human stone, the method includes preventing activation of the first laser 16.

The method may further include moving the distal end 32 of the waveguide 30 until a second human stone 40b is located within the treatment space 80, as shown in FIG. 4. When the second human stone 40b is located within the treatment space 80, the focused beam of light 52 impacts the second human stone 40b causing a fluorescence signal 54 to be emitted by the second human stone 40b. The fluorescence signal 54 is captured by the waveguide 30 and guided to the optical detector 56, which identifies the fluorescence signal 54 as being emitted by a human stone. Upon identification of the second human stone 40b within the treatment space 80, the first laser 16 is activated producing the focused beam of light 18, which is guided to the second human stone 40b.

According to one embodiment, the first laser 16 includes multiple modes of activation (e.g., pulsed or continuous wave). The method may include selecting the second mode and activating the first laser 16 to produce intermittent pulses of the focused beam of light 18.

The above description of illustrated embodiments, including what is described in the Abstract, is not intended to be exhaustive or to limit the embodiments to the precise forms disclosed. Although specific embodiments of and examples are described herein for illustrative purposes, various equivalent modifications can be made without departing from the spirit and scope of the disclosure, as will be recognized by those skilled in the relevant art.

Many of the methods described herein can be performed with variations. For example, many of the methods may include additional acts, omit some acts, and/or perform acts in a different order than as illustrated or described. The various embodiments described above can be combined to provide further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A laser lithotripsy system comprising:
a first laser that, upon activation, produces laser light with a first wavelength, wherein the first laser includes a first activation mode and a second activation mode, when the first laser is in the first activation mode the first laser produces a continuous wave of laser light with the first wavelength, and when the first laser is in the second activation mode the first laser produces uniformly spaced, intermittent pulses of laser light with the first wavelength;
a second laser that, upon activation, produces laser light with a second wavelength, which is shorter than the first wavelength;
a first optically powered element positioned to receive both the laser light from the first laser and the laser light from the second laser, wherein the optically powered surface transmits at least 90% of the laser light received from the first laser and reflects at least 90% of the laser light received from the second laser such that the transmitted laser light from the first laser and the reflected laser light from the second laser are superimposed;
a waveguide positioned to receive the coincidental laser light from the first and second lasers and guide the superimposed laser light to a target;
an optical detector positioned to receive light emitted by the target and measure one or more characteristics of the received light emitted by the target;
a controller communicatively coupled to both the optical detector and the first laser such that the controller allows activation of the first laser to produce the continuous wave of laser light with the first wavelength only when the one or more measured characteristics are within a predetermined range of values.

2. The laser lithotripsy system of claim 1 wherein the controller is communicatively coupled to both the optical detector and the first laser such that the controller prevents activation of the first laser when the one or more measured characteristics are outside of the predetermined range of values.

3. The laser lithotripsy system of claim 1 wherein the first laser produces light with a wavelength of less than 2100 nm.

4. The laser lithotripsy system of claim 1 wherein the first laser produces light with a wavelength of less than 2050 nm.

5. The laser lithotripsy system of claim 1 wherein the first laser produces light with a wavelength of less than 2000 nm.

6. The laser lithotripsy system of claim 1 wherein the first laser is a thulium-based laser.

7. The laser lithotripsy system of claim 1 further comprising:
a housing enclosing the first laser, the second laser, the optical detector, and the controller.

8. The laser lithotripsy system of claim 7 wherein the housing is mounted on one or more wheels.

9. The laser lithotripsy system of claim 1, further comprising:
a second optically powered surface positioned to receive the light emitted by the target, wherein the light emitted by the target has a third wavelength, which is shorter than the first wavelength and longer than the second wavelength.

10. The laser lithotripsy system of claim 9 wherein the second optically powered surface reflects at least 90% of the light emitted by the target and received by the second optically powered surface.

11. The laser lithotripsy system of claim 1 wherein the light emitted by the target has a wavelength between 550 nm and 900 nm.

12. The laser lithotripsy system of claim 1 wherein the second laser is a green excitation laser.

13. The laser lithotripsy system of claim 12 wherein the second wavelength is between 520 nm and 532 nm.

14. A method of operating a laser lithotripsy system, the method comprising:
activating an excitation laser to produce laser light and guiding the produced laser light to a target via a waveguide;
capturing light emitted from the target as a result of the laser light produced by the excitation laser impacting the target;
guiding the captured light emitted from the target to an optical detector via the waveguide;
measuring one or more characteristics of the captured light emitted by the target and guided to the optical detector;
comparing the one or more measured characteristics to a predetermined set of values for each of the one or more measured characteristics; and
activating a therapeutic laser when the one or more measured characteristics are within the respective predetermined set of values for each of the one or more measured characteristics, wherein activating the therapeutic laser produces a continuous wave of laser light when the therapeutic laser is in a first activation mode, and activating the therapeutic laser produces uniformly spaced, intermittent pulses of laser light when the therapeutic laser is in a second activation mode.

15. The method of claim 14 wherein the therapeutic laser is a thulium-based laser that produces laser light having a wavelength of between 1800 nm and 2200 nm.

16. The method of claim 14, further comprising:
deactivating the therapeutic laser when the one or more measured characteristics are outside the respective predetermined set of values for each of the one or more measured characteristics.

17. The method of claim 14 wherein measuring the one or more characteristics includes measuring an amplitude of the captured light emitted by the target and guided to the optical detector.

18. The method of claim 14 wherein measuring the one or more characteristics includes measuring spectroscopic information of the captured light emitted by the target and guided to the optical detector to determine a chemical makeup of the target.

19. The method of claim 14, further comprising:
identifying the target based on the one or more measured characteristics.

20. The method of claim 14, further comprising:
enclosing the excitation laser, the therapeutic laser, and the optical detector within a housing; and
moving the housing from a first location to a second location.

21. The method of claim 14, further comprising:
transitioning the therapeutic laser from one of the first activation mode and the second activation mode to the other of the first activation mode and the second activation mode.

22. A method of treating human stones, the method comprising:
activating an excitation first laser to produce laser light and guiding the produced laser light to a distal end of a waveguide where the produced laser light exits the waveguide;
moving the distal end of the waveguide such that the produced laser light exits the waveguide and impacts a human stone;
capturing light emitted from the human stone as a result of the laser light produced by the excitation laser impacting the human stone;
guiding the captured light emitted from the human stone to an optical detector via the waveguide;
measuring one or more characteristics of the captured light emitted by the human stone and guided to the optical detector;
determining whether the captured light was emitted by a human stone based on the measured one or more characteristics of the captured light;
after determining the captured light was emitted by a human stone, activating a therapeutic laser thereby producing either a continuous wave of laser light with a first wavelength or uniformly spaced, intermittent pulses of laser light with the first wavelength; and
guiding the laser light with the first wavelength to the distal end of the waveguide where the laser light with the first wavelength exits the waveguide and impacts the human stone.

23. The method of claim 22, further comprising:
guiding the laser light with the first wavelength to impact the human stone until the human stone breaks into multiple fragments.

24. The method of claim 23, further comprising:
after breaking the human stone into multiple fragments, moving one or more of the fragments, the distal end of the waveguide, or both one or more of the fragments and the distal end of the waveguide such that the laser light produced by the excitation laser exits the waveguide and impacts a target other than one of the multiple fragments of the human stone;
capturing light emitted from the target as a result of the light produced by the excitation laser impacting the target;
guiding the captured light emitted from the target to the optical detector via the waveguide;
measuring one or more characteristics of the captured light emitted by the target and guided to the optical detector;
determining whether the captured light emitted by the target was emitted by a human stone based on the measured one or more characteristics of the captured light emitted by the target;
after determining the captured light emitted by the target was not emitted by a human stone, deactivating the therapeutic laser.

25. The method of claim 24 wherein the human stone is a first human stone, the method further comprising:
moving the target, the distal end of the waveguide, or both the target and the distal end of the waveguide such that the laser light produced by the excitation laser exits the waveguide and impacts a second human stone;
capturing light emitted from the second human stone as a result of the laser light produced by the excitation laser impacting the second human stone;
guiding the captured light emitted from the second human stone to the optical detector via the waveguide;
measuring one or more characteristics of the captured light emitted by the second human stone and guided to the optical detector;
determining whether the captured light emitted by the second human stone was emitted by a human stone based on the measured one or more characteristics of the captured light emitted by the second human stone;
after determining the captured light emitted by the second human stone was emitted by a human stone, reactivating the therapeutic laser; and
guiding the laser light produced by the reactivated therapeutic laser to the distal end of the waveguide where the laser light produced by the reactivated therapeutic laser exits the waveguide and impacts the second human stone.

26. The method of claim 22 wherein the laser light produced by the therapeutic laser has a wavelength of less than 2100 nm.

27. The method of claim 22 wherein each of: the excitation laser, the therapeutic laser, and the optical detector are enclosed within a housing.

28. The method of claim 22 wherein the therapeutic laser includes a first activation mode in which the therapeutic laser produces the continuous wave of laser light with the first wavelength, and a second activation mode in which the therapeutic laser produces the uniformly spaced, intermittent pulses of laser light with the first wavelength, the method further comprising:
transitioning the therapeutic laser from one of the first activation mode and the second activation mode to the other of the first activation mode and the second activation mode.
